(19) United States
(12) Patent Application Publication
(10) Pub. No.: US 2024/0065366 A1
(43) Pub. Date: Feb. 29, 2024

(54) VOICE-CONTROLLED WELDING CAP

(71) Applicant: NINGBO JIALANG INTELLIGENT TECHNOLOGY CO.,LTD., Yuyao (CN)

(72) Inventor: HAIBING WU, Yuyao (CN)

(21) Appl. No.: 17/987,046

(22) Filed: Nov. 15, 2022

(30) Foreign Application Priority Data
Aug. 25, 2022 (CN) 202211026608.2

(51) Int. Cl. A42B 3/30; G10L 15/22; G10L 15/28; H04R 1/02; H04R 1/08; H04R 3/00

(52) U.S. Cl. CPC A42B 3/30; G10L 15/22; G10L 15/28; H04R 1/028; H04R 1/08; H04R 3/00; G02F 1/133308

(57) ABSTRACT

The present disclosure discloses a voice-controlled welding cap, which comprises a welding cap body, wherein the outer side of the welding cap body is provided with a mask, the inner side of the welding cap body is provided with a panel frame, a head ring, a connecting plate and an optical sensor, the inner side of the panel frame is provided with a main control chip and a language module input unit, and the language module input unit comprises a microphone, a voice chip and a peripheral resistance-capacitance device; the main control chip uses an STC8H 8BIT microprocessor control unit, the microphone is connected with the voice chip, and the voice chip, the mask and the optical sensor are all connected with the main control chip.

### CROSS-REFERENCE TO RELATED APPLICATIONS

This is a U. S. patent application which claims the priority and benefit of Chinese Patent Application Number 202211026608.2, filed on Aug. 25, 2022, the disclosure of which is incorporated herein by reference in its entirety.

### TECHNICAL FIELD

The present disclosure belongs to the technical field of welding caps, in particular to a voice-controlled welding cap.

### BACKGROUND

Most of the existing digital masks need to manually adjust the color number when adjusting parameters. If the time delay and sensitivity are adjusted, the mask needs to be removed for adjustment. The adjustment method is still very inconvenient and safe. For example, hands need to be free for operation. Sometimes, in order to adjust the appropriate parameters, the mask needs to be taken off many times, which is very tedious. For another example, when working at heights, it is very unsafe to often operate the mask.

### SUMMARY

The purpose of the present disclosure is to provide a voice-controlled welding cap, which facilitates the adjustment of common parameters of a welding cap by welding workers in the use process and improves the safety of welding workers,

In order to achieve the above purpose, the present disclosure provides the following technical scheme. A voice-controlled welding cap is provided, which comprises a welding cap body, wherein the outer side of the welding cap body is provided with a mask, the inner side of the welding cap body is provided with a panel frame, a head ring, a connecting plate and an optical sensor, the inner side of the panel frame is provided with a main control chip and a language module input unit, and the language module input unit comprises a microphone, a voice chip and a peripheral resistance-capacitance device; the main control chip uses an STC8H 8BIT microprocessor control unit, the microphone is connected with the voice chip, and the voice chip, the mask and the optical sensor are all connected with the main control chip. The main control chip is mainly responsible for the main control of the whole product, issuing a liquid crystal signal for controlling the mask, outputting a pulse drive signal with a certain frequency through an MCU timer, detecting the external signal in real time, and using the external interrupt detection function of the MCU. The main control chip can quickly detect strong light stimulation, make an output response quickly, change the contrast of the liquid crystal, and effectively avoid the strong light stimulation to human eyes.

As a preferred technical scheme of the present disclosure, the voice chip uses a 32-bit RISC core chip, supports an MIC input, supports AGC function, and has a built-in voice integrated library for offline identification, which can identify Chinese and English.

As a preferred technical scheme of the present disclosure, the voice-controlled welding cap further comprises an amplifier module, which transmits the audio signal received by the microphone to the language chip to amplify the signal therein.

As a preferred technical scheme of the present disclosure, the voice-controlled welding cap further comprises an acquisition and conversion module, which is used to realize data acquisition and conversion and output corresponding data signals.

As a preferred technical scheme of the present disclosure, the language chip and the main control chip are connected by a two-wire serial data line.

As a preferred technical scheme of the present disclosure, the panel frame is provided with an LCD display screen, and the main control chip is provided with an LCD hardware interface, so that the LCD display screen can be directly lit without any driver chip.

As a preferred technical scheme of the present disclosure, the panel frame is provided with a key, and the key is connected with the main control chip, so that the key scanning can be processed in real time, and the key scanning carries out the corresponding action after reading the key value in a timing scanning manner.

As a preferred technical scheme of the present disclosure, the panel frame is provided with a TYPE-C interface for charging, and a rechargeable lithium battery pack is provided inside the panel frame. Generally, most of these products use non-rechargeable batteries, and when the power is exhausted, it is troublesome to buy batteries. The present disclosure uses a rechargeable lithium battery pack, uses a mobile phone TYPE-C interface, and has long battery life, which is convenient to charge. Moreover, the present disclosure has a full-charge protection function and a corresponding full-charge indicator light, which is very convenient to use.

Compared with the prior art, the present disclosure has the following beneficial effect.
1. The present disclosure greatly facilitates the adjustment of common parameters of a welding cap by welding workers in the use process, improves the safety of welding workers, and simultaneously improves the efficiency of welding operation.
2. As a TYPE-C charging mode is used, the use cost is reduced, and the service life of the welding cap is prolonged to some extent.

### BRIEF DESCRIPTION OF DRAWINGS

FIG. 1 is a control chart of the present disclosure.

FIG. 2 is a schematic structural diagram of the present disclosure.

FIG. 3 is a system diagram of the present disclosure.

In the figures: 1. Mask; 4, Welding cap body; 5. Panel frame; 11. Head ring; 18. Connecting plate; 19. Optical sensor.

### DETAILED DESCRIPTION

The technical scheme in the embodiments of the present disclosure will be clearly and completely described with reference to the drawings in the embodiments of the present disclosure hereinafter. Obviously, the described embodiments are only some embodiments of the present disclosure, rather than all of the embodiments. Based on the embodiment of the present disclosure, all other embodiments obtained by those skilled in the art without creative labor fall within the scope of protection of the present disclosure.

### Embodiment 1

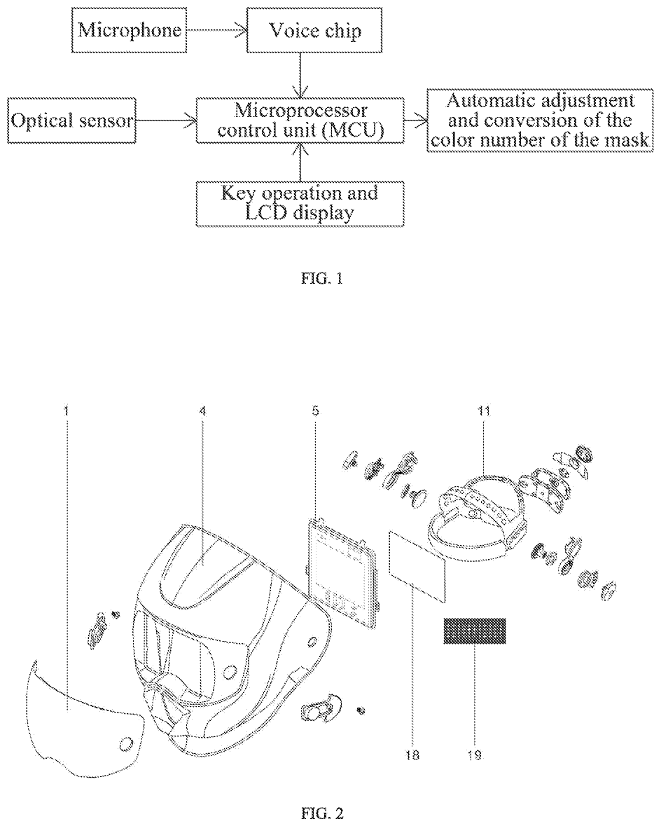

Refer to FIG. 2, which is embodiment 2 of the present disclosure. This embodiment provides a voice-controlled welding cap, comprising a welding cap body 4, wherein the outer side of the welding cap body 4 is provided with a mask 1, so that the mask 1 and the welding cap body 4 are spliced. The inner side of the welding cap body 4 is provided with a panel frame 5, a head ring 11, a connecting plate 18 and an optical sensor 19, so as to install the panel frame 5, the head ring 11, the connecting plate 18 and the optical sensor 19.

### Embodiment 2

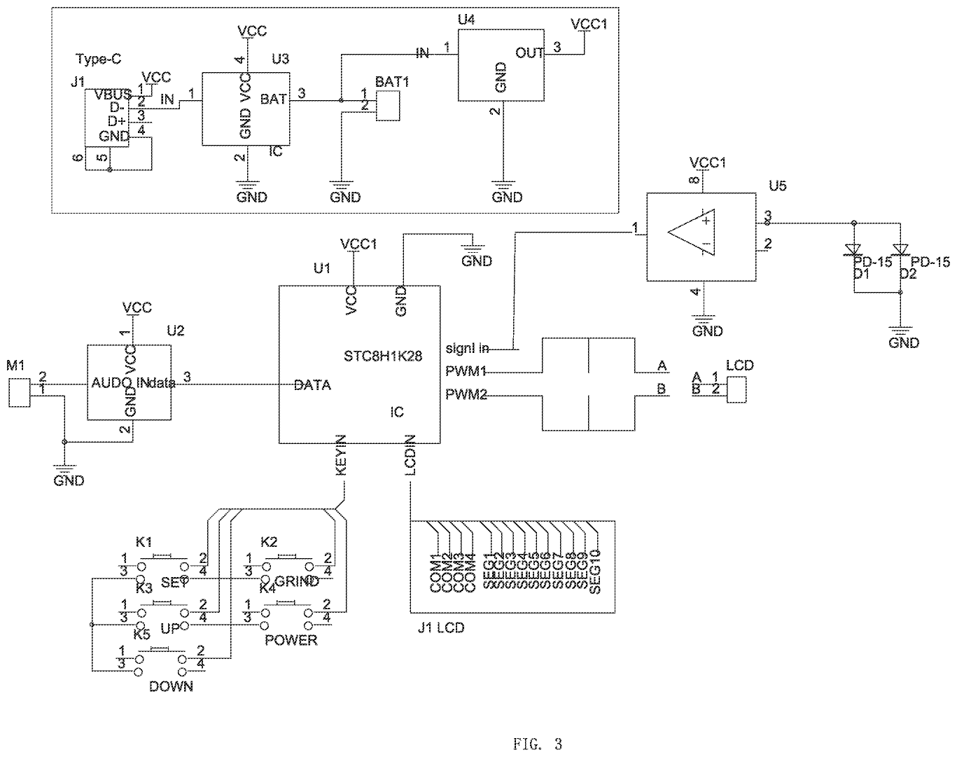

Refer to FIGS. 1, 2 and 3, which are embodiment 2 of the present disclosure. This embodiment is based on the previous embodiment, and the difference is as follows.

The inner side of the panel frame 5 is provided with a main control chip and a language module input unit, and the language module input unit comprises a microphone, a voice chip and a peripheral resistance-capacitance device; the main control chip uses an STC8H 8BIT microprocessor control unit, the microphone is connected with the voice chip, and the voice chip, the mask 1 and the optical sensor 19 are all connected with the main control chip, which are as follows.

The voice chip uses a high-performance 32-bit RISC core chip, supports an MIC input, supports AGC function, and has a variety of built-in voice integrated libraries for offline identification, which can identify Chinese, English and other languages. After the microphone receives the audio signal, the audio signal is transmitted to the inside of the chip. After internal signal amplification, data acquisition and conversion, the corresponding data signal is output, and the command data is output through a two-wire serial data line.

The main control chip uses an STC8H 8BIT microprocessor control unit, which is mainly responsible for the main control of the whole product. First, the command data sent by the language module input unit parses the corresponding the command using the RS232 serial protocol at a rate of 9600. Second, the key scanning and the LCD display are processed in real time. The key scanning carries out the corresponding action after reading the key value in a timing scanning manner. The main control chip has its own LCD hardware interface, which can directly light the LCD display without any driver chip. Third, a liquid crystal signal for controlling the mask is issued, and a pulse drive signal with a certain frequency is output through an Mai timer. Fourth, the external signal is detected in real time, and the external interrupt detection function of the MCU is used. The main control chip can quickly detect strong light stimulation, make an output response quickly, change the contrast of the liquid crystal, and effectively avoid the strong light stimulation to human eyes.

The liquid crystal of the mask 1 is controlled. The working principle is a liquid crystal light valve controlled by power. When there is no power, the liquid crystal light valve is transparent. After power is turned on, the liquid crystal light valve immediately turns into a black screen, thus blocking strong light or weakening the intensity of light. The liquid crystal valve is an optical switch, which switches from transparence to opacity. The main control chip issues the signal with a certain frequency and a certain voltage amplitude for control.

Generally, most of these products use non-rechargeable batteries, and when the power is exhausted, it is troublesome to buy batteries. The present disclosure uses a rechargeable lithium battery pack, uses a mobile phone TYPE-C interface, and has long battery life, which is convenient to charge. Moreover, the present disclosure has a full-charge protection function and a corresponding full-charge indicator light, which is very convenient to use.

The corresponding software is controlled as follows.

The optical signal input is detected, and the color number of the mask starts to be controlled.

The timing interruption needs to judge whether there is an optical signal, if so, the driving pulse is output, otherwise, the driving pulse is turned off, and the working flag bit is accumulated by a timer.

The main function judges the key scanning flag. If so, the key processing function is used for processing. Otherwise, it is judged whether there is a voice signal. If so, the voice command processing function is used. Otherwise, the LCD refresh flag is displayed. The LCD refresh flag is judged. If so, the content refresh function is displayed.

Although the embodiments of the present disclosure have been shown and described, as detailed in the above detailed description. It will be understood by those skilled in the art that many changes, modifications, substitutions and variations can be made to these embodiments without departing from the principle and spirit of the present disclosure, and the scope of the present disclosure is defined by the appended claims and their equivalents.

What is claimed is:

1. A voice-controlled welding cap, comprising a welding cap body (4), wherein the outer side of the welding cap body (4) is provided with a mask (1), the inner side of the welding cap body (4) is provided with a panel frame (5), a head ring (11), a connecting plate (18) and an optical sensor (19), the inner side of the panel frame (5) is provided with a main control chip and a language module input unit, and the language module input unit comprises a microphone, a voice chip and a peripheral resistance-capacitance device; the main control chip uses an STC8H 8BIT microprocessor control unit, the microphone is connected with the voice chip, and the voice chip, the mask (1) and the optical sensor (19) are all connected with the main control chip.

2. The voice-controlled welding cap according to claim 1, wherein the voice chip uses a 32-bit RISC core chip, supports an MIC input, supports AGC, and has a built-in voice integrated library for offline identification.

3. The voice-controlled welding cap according to claim 1, further comprising an amplifier module, which transmits the audio signal received by the microphone to the language chip to amplify the signal therein.

4. The voice-controlled welding cap according to claim 1, further comprising an acquisition and conversion module, which is used to realize data acquisition and conversion and output corresponding data signals.

5. The voice-controlled welding cap according to claim 1, wherein the language chip and the main control chip are connected by a two-wire serial data line.

6. The voice-controlled welding cap according to claim 1, wherein the panel frame (5) is provided with an LCD display screen, and the main control chip is provided with an LCD hardware interface.

7. The voice-controlled welding cap according to claim 1, wherein the panel frame (5) is provided with a key, and the key is connected with the main control chip.

8. The voice-controlled welding cap according to claim 1, wherein the panel frame (5) is provided with a TYPE-C interface for charging, and a rechargeable lithium battery pack is provided inside the panel frame (5).
